(19) United States
(12) Patent Application Publication
(10) Pub. No.: US 2023/0145329 A1
(43) Pub. Date: May 11, 2023

(54) ARTHROPLASTY IMPLANT SYSTEMS WITH STEMLESS IMPLANTS

(71) Applicant: ARTHREX, INC., Naples, FL (US)

(72) Inventors: John David PATERSON, Naples, FL (US); Michael Alan KNIGHT, Naples, FL (US)

(21) Appl. No.: 18/054,165

(22) Filed: Nov. 10, 2022

Related U.S. Application Data
(60) Provisional application No. 63/278,226, filed on Nov. 11, 2021.

(51) Int. Cl. A61F 2/40; A61F 2/30

(52) U.S. Cl. CPC A61F 2/4003; A61F 2/30756; A61F 2/30734; A61F 2/30771; A61F 2002/3069; A61F 2002/3085; A61F 2002/30474; A61F 2002/30481; A61F 2002/30235; A61F 2002/30327; A61F 2002/3093; A61F 2002/30461; A61F 2002/30471; A61F 2002/30574

(57) ABSTRACT

Arthroplasty implant systems and methods are provided for restoring the functionality of a joint. The arthroplasty implant systems may include a threaded cup having a cylindrical shaped body and a flange. A thread may be provided on the cylindrical shaped body. The thread is configured to engage a bone, and the flange is configured to engage a cortical rim of the bone.

### CROSS-REFERENCE TO RELATED APPLICATIONS

This disclosure claims priority to U.S. Provisional Application No. 63/278,226, filed on Nov. 11, 2021, the entire disclosure of which is incorporated herein by reference.

### BACKGROUND

This disclosure relates to the field of arthroplasty, and more particularly to arthroplasty implant systems that include implants capable of establishing a stemless convertible platform for interfacing with articular implants.

Many bones of the human musculoskeletal system include articular surfaces. The articular surfaces cooperate to facilitate different types and degrees of joint movement. The articular surfaces can erode or experience bone loss over time due to repeated use or wear, thereby causing joint instability and pain.

Arthroplasty is an orthopedic surgical procedure performed to repair or replace joints that exhibit degenerative bone deficiencies. Bone deficiencies may occur along the articular surfaces of bone. Some arthroplasty procedures utilize one or more implants to repair the articular surfaces.

### SUMMARY

This disclosure relates to arthroplasty implant systems and methods designed for restoring functionality to a joint. The arthroplasty implant systems may include an implant assembly that includes a stemless convertible implant.

An exemplary humeral implant assembly for an arthroplasty implant system may include, inter alia, an articular implant, and a stemless implant adapted to establish a convertible platform for receiving the articular implant. The stemless implant includes a thread configured to engage a cortical and/or cancellous bone of a bone and a flange sized to engage a cortical rim of the bone.

In a further embodiment, the articular implant is an anatomic articular implant that includes a convex articular surface.

In a further embodiment, the articular implant is a reverse articular implant that includes a concave articular surface.

In a further embodiment, the articular implant includes a spacer that is coupled to the stemless implant and a liner that is coupled to the spacer. The liner includes a concave articular surface.

In a further embodiment, the spacer is coupled to the stemless implant by a C-ring.

In a further embodiment, the stemless implant is comprised of a polyether ether ketone (PEEK) material.

In a further embodiment, the stemless implant includes a receiving cavity adapted to receive the articular implant, and the receiving cavity extends inwardly from the flange to a floor of a rounded base of the stemless implant.

In a further embodiment, the floor establishes an inner surface of the rounded base.

In a further embodiment, the thread is circumferentially disposed about a radially outer surface of a cylindrical shaped body of the stemless implant.

In a further embodiment, an outer diameter of the flange is greater than an outer diameter of the cylindrical shaped body at a tip of the thread.

In a further embodiment, the cylindrical shaped body includes a plurality of pockets adapted to facilitate bony ingrowth.

In a further embodiment, the flange includes a plurality of suture eyelets that are each configured to receive a thread-like material.

An exemplary arthroplasty implant system may include, inter alia, a threaded cup having a cylindrical shaped body and a flange. A thread may be provided on the cylindrical shaped body. The thread is configured to engage cortical and/or cancellous bone of a bone, and the flange is sized to engage a cortical rim of the bone.

In a further embodiment, the flange is integrally formed with the cylindrical shaped body to establish a unitary single piece design of the threaded cup.

In a further embodiment, the threaded cup embodies an inlay design that establishes a convertible platform for receiving an articular implant.

In a further embodiment, the articular implant includes a spacer coupled to the threaded cup by a C-clip, and a liner coupled to the spacer.

In a further embodiment, the flange is provided on a first side of the cylindrical shaped body and a rounded base is provided on a second side of the cylindrical shaped body.

In a further embodiment, a receiving cavity extends inwardly from the flange to a floor of the rounded base, and further wherein the floor establishes an inner surface of the rounded base.

In a further embodiment, at least one engagement opening is formed through the rounded base of the threaded cup.

In a further embodiment, the rounded based includes a trap door.

In a further embodiment, an outer diameter of the flange is greater than an outer diameter of the cylindrical shaped body at a tip of the thread.

In a further embodiment, the cylindrical shaped body includes a plurality of pockets adapted to facilitate bony ingrowth.

In a further embodiment, the flange includes a plurality of suture eyelets that are each configured to receive a thread-like material.

### BRIEF DESCRIPTION OF THE DRAWINGS

FIG. 1 illustrates a humeral implant assembly of an arthroplasty implant system.

FIG. 2 illustrates another exemplary humeral implant assembly of an arthroplasty implant system.

FIG. 3 illustrates an exemplary threaded cup of a humeral implant assembly.

FIG. 4 is a top perspective view of the threaded cup of FIG. 3.

FIG. 5 is a side perspective view of the threaded cup of FIG. 3.

FIG. 6 illustrates a flange of the threaded cup of FIG. 3 engaging a cortical rim of a bone.

FIG. 7 illustrates various details associated with a circumferential thread of the threaded cup of FIGS. 3-6.

FIG. 8 illustrates another exemplary threaded cup of a humeral implant assembly.

FIG. 9 illustrates a modular stem that can be utilized with the threaded cup of FIG. 8.

FIG. 10 illustrates a humeral implant assembly that includes a threaded cup.

FIG. 11 is an exploded view of the humeral implant assembly of FIG. 10.

FIG. 12 is a cross-sectional view of the humeral implant assembly of FIG. 10.

FIGS. 13A and 13B illustrate a spacer of another exemplary humeral implant assembly.

FIG. 14 illustrates a liner of the humeral implant assembly of FIG. 10.

FIG. 15 illustrates another exemplary liner of the humeral implant assembly of FIG. 10.

### DETAILED DESCRIPTION

This disclosure describes arthroplasty implant systems and methods for restoring the functionality of a joint. The arthroplasty implant systems may include implants capable of establishing a stemless convertible platform for interfacing with articular implants.

In some implementations, the arthroplasty implant systems of this disclosure may include a threaded cup having a cylindrical shaped body and a flange. A thread may be provided on the cylindrical shaped body. The thread is configured to engage cortical and/or cancellous bone of a bone, and the flange is configured to engage a cortical rim of the bone. These and other features of this disclosure are further detailed below.

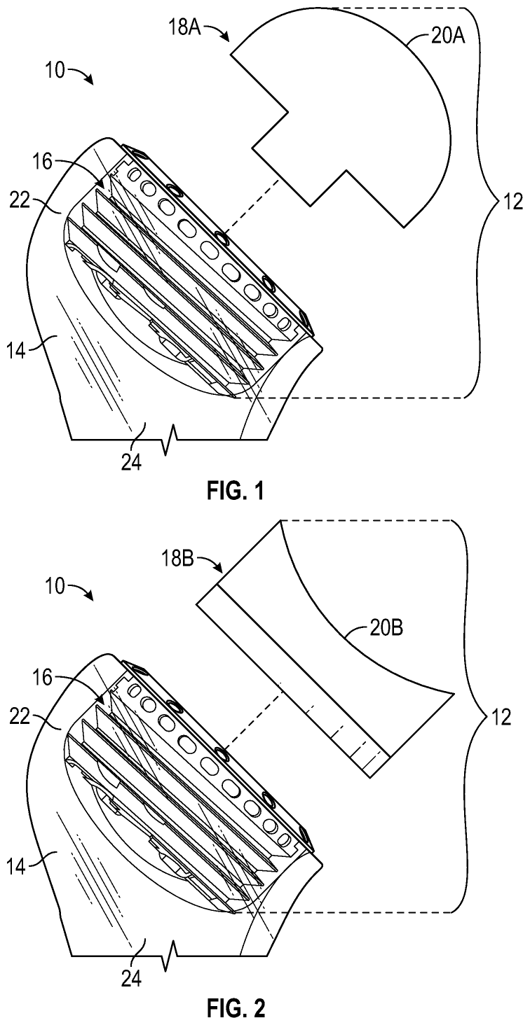

FIG. 1 illustrates an arthroplasty implant system 10 that includes a humeral implant assembly 12. The humeral implant assembly 12 may be implanted within a humerus 14 of a shoulder joint to aid in reconstructing the native articular surface of the humerus 14 and/or restoring the functionality (e.g., range or motion, stability, etc.) of the shoulder joint. Although not shown, the arthroplasty implant system 10 could additionally include a glenoid implant assembly that is configured to interface with the humeral implant assembly 12 for restoring functionality to the shoulder joint. Moreover, although the teachings of this disclosure are described with specific reference to the shoulder joint, this disclosure is not intended to be limited to any particular joint of the human musculoskeletal system and could be applicable to other joints, such as the hip joint, for example.

The humeral implant assembly 12 may include a threaded cup 16 and an anatomic articular implant 18A (see FIG. 1) or a reverse articular implant 18B (see FIG. 2). The anatomic articular implant 18A may be utilized in combination with the threaded cup 16 for performing anatomic total shoulder arthroplasty procedures, and the reverse articular implant 18B may be utilized in combination with the threaded cup 16 for performing reverse shoulder arthroplasty procedures. In reverse shoulder arthroplasty procedures, the reconstituted humerus 14 provides the socket portion and the glenoid (not shown) provides the ball portion of the ball-and-socket joint, which is the opposite of the native anatomy. The anatomic articular implant 18A may include a convex articular surface 20A, and the reverse articular implant 18B may include a concave articular surface 20B. The articular surfaces 20A, 20B are configured to interface with the native glenoid or a glenoid implant assembly of the arthroplasty implant system 10.

In some implementations, the threaded cup 16, the anatomic articular implant 18A, and the reverse articular implant 18B may be provided together as part of a surgical kit. The surgical kit could additionally could multiple sizes of each of the threaded cup 16, the anatomic articular implant 18A, and the reverse articular implant 18B.

In the illustrated embodiment, a humeral head of the humerus 14 has been resected, and thus the native articular component of the humerus 14 is removed in order to prepare the humerus 14 for receiving the humeral implant assembly 12. After the humerus 14 has been appropriately prepared, the threaded cup 16 may be screwed into a metaphysis 22 of the humerus 14. In an embodiment, the threaded cup 16 is a stemless implant of the humeral implant assembly 12 and therefore lacks a stem that extends into a diaphysis 24 of the humerus 14. The threaded cup 16 may be configured to establish a convertible platform for receiving either the anatomic articular implant 18A or the reverse articular implant 18B. The anatomic articular implant 18A or the reverse articular implant 18B may be mounted to the threaded cup 16 for assembling the humeral implant assembly 12.

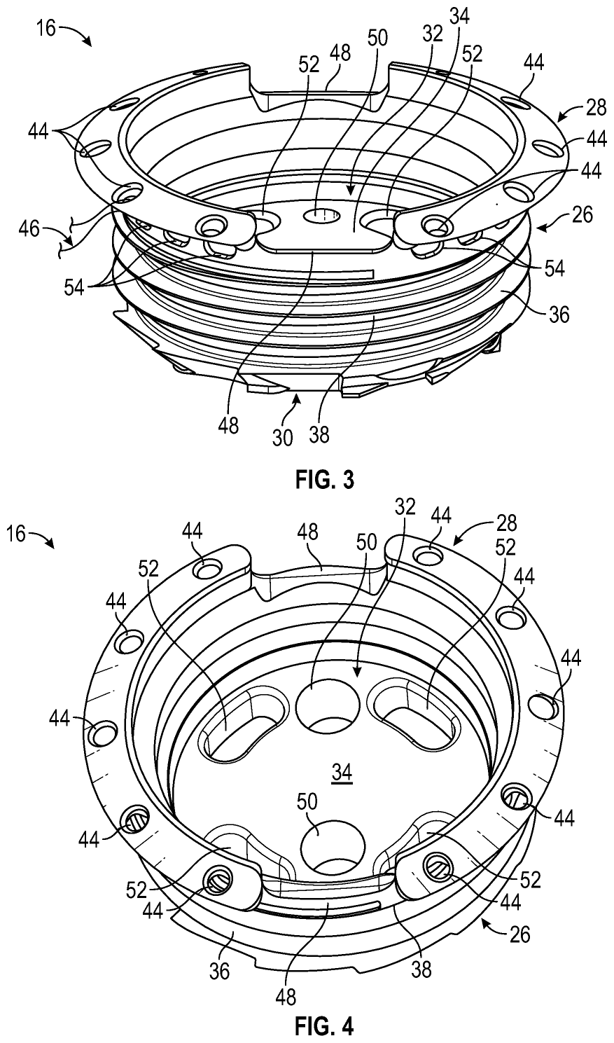

The threaded cup 16 of the humeral implant assembly 12 is further illustrated in FIGS. 3, 4, and 5 (with continued reference to FIGS. 1 and 2). In an embodiment, the threaded cup 16 is constructed of a titanium material. In another embodiment, the threaded cup 16 may be made of a material that has an elastic modulus that is relatively close to that of cortical bone, such as polyether ether ketone (PEEK), for example. Other materials for constructing the threaded cup 16 could alternatively be utilized within the scope of this disclosure.

The threaded cup 16 may include a cylindrical shaped body 26 that extends between a flange 28 located at a top or proximal side of the threaded cup 16 and a rounded base 30 located at a bottom or distal side of the threaded cup 16. In this embodiment, the threaded cup 16 embodies a unitary single piece design in which the flange 28 is integrally formed with the cylindrical shaped body 26.

A receiving cavity 32 of the threaded cup 16 may be configured to receive and secure either the anatomic articular implant 18A or the reverse articular implant 18B to the threaded cup 16. The receiving cavity 32 may be circumscribed by the flange 28 and the cylindrical shaped body 26. The receiving cavity 32 may extend inwardly from the flange 28 to a floor 34 of the rounded base 30. The floor 34 may establish an inner surface of the rounded base 30.

The receiving cavity 32 may provide an inlay design in which a majority of the threaded cup 16 (with the exception of the flange 28) is disposed inside the humerus 14. In this way, the connection between the threaded cup 16 and the articular implant 18A, 18B is also inlaid rather than exhibiting an onlay design.

A thread 36 may be circumferentially disposed about a radially outer surface 38 of the cylindrical shaped body 26. The thread 36 may be a self-tapping thread configured to allow the threaded cup 16 to be screwed into the humerus 14. The thread 36 may be configured such that either a clockwise rotation or a counterclockwise rotation functions to advance the threaded cup 16 into the humerus 14.

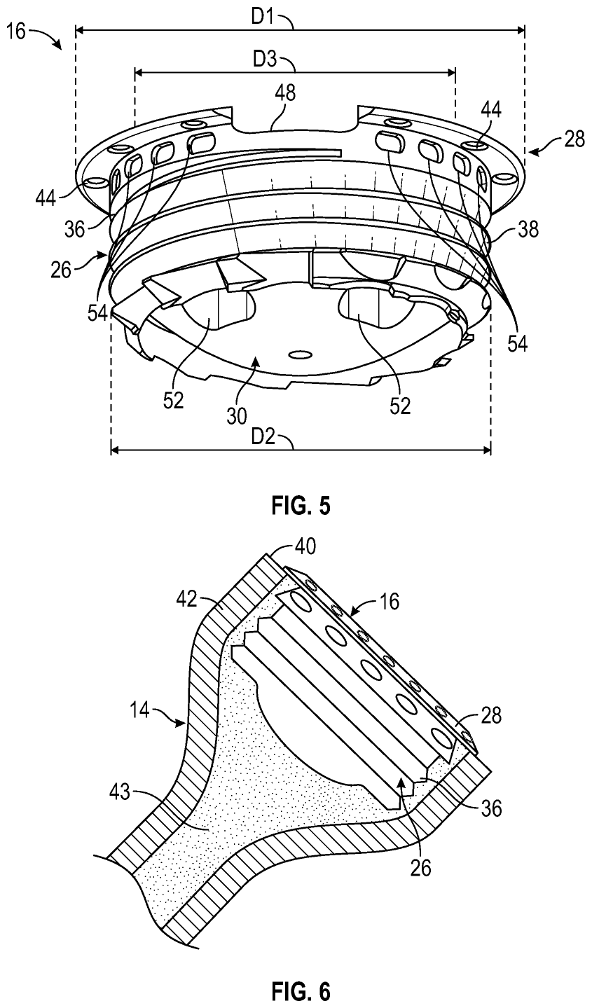

In an embodiment, an outer diameter D1 of the flange 28 is greater than an outer diameter D2 (defined here at a tip of the thread 36) of the cylindrical shaped body 26 (see, e.g., FIG. 5). The outer diameter D1 of the flange 28 may be sized to enable the flange 28 to engage a cortical rim 40 (see, e.g., FIG. 6) of the humerus 14. Engaging the cortical rim 40 in this manner may provide additional fixation support and load the proximal portion of the humerus 14 more favorably.

Moreover, the outer diameter D2 may be sized such that the thread 36 is positioned in relatively close proximity to cortical bone 42 of the humerus 14 once the threaded cup 16 is inserted therein (see, e.g., FIG. 6). Fixating the threaded cup 16 with the thread 36 as close to the cortical bone 42 as possible may provide improved initial and long-term fixation as compared to “press-fit” implant designs. The thread 36 may thus engage cortical bone 42, cancellous bone 43, or both.

The cylindrical shaped body 26 may further include an inner diameter D3. The inner diameter D3 may establish a cup size of the threaded cup 16. The inner diameter D3 may be less than both the outer diameter D1 and the outer diameter D3.

The actual dimensions of the outer diameter D1 of the flange 28, the outer diameter D2 of the cylindrical shaped body 26, and the inner diameter D3 of the cylindrical shaped body 26 may vary depending on the size of the patient, among other factors. The surgical kit referenced above could include threaded cups having multiple combinations of flange 28 outer diameter sizes and cylindrical shaped body 26 inner diameter sizes. Table 1 below illustrates exemplary sizes of threaded cups 16 that could be provided as part of the surgical kit. The listed sizes are exemplary only and thus intended to be non-limiting.

TABLE 1
Exemplary Sizes of Threaded Cup 16

| OD of | ID of Threaded Cup 16 | | | | |
|---|---|---|---|---|---|
| Flange 28 | 30 mm | 33 mm | 36 mm | 39 mm | 42 mm |
| 41 mm | ● | | | | |
| 43 mm | ● | | | | |
| 45 mm | ● | ● | | | |
| 47 mm | ● | ● | ● | | |
| 49 mm | ● | ● | ● | | |
| 51 mm | ● | ● | ● | ● | |
| 53 mm | ● | ● | ● | ● | |
| 55 mm | ● | ● | ● | ● | ● |

The flange 28 of the threaded cup 16 may be either circular or elliptical shaped. However, the actual shape of the flange 28 is not intended to limit this disclosure.

A plurality of suture eyelets 44 may extend through the flange 28. The suture eyelets 44 may be configured to receive a thread-like material, such as a suture 46 (see FIG. 3). The suture 46 may then be utilized to assist with tying tissue (e.g., subscapularis muscle, supraspinatus muscle, etc.) to the humerus 14 in the area around the flange 28.

One or more cutouts or interruptions 48 may be formed in the flange 28/cylindrical shaped body 26 of the threaded cup 16. The interruptions 48 are sized to receive mating features of a wedge/spacer (not shown) that may be utilized in combination with the threaded cup 16 for reducing laxity between the threaded cup 16 and the articular implant 18A, 18B. The mating features of the wedge/spacer may engage walls of the flange 28 that delineate the interruptions 48 to prevent rotation of the wedge/spacer relative to the threaded cup 16.

One or more engagement openings 50 may be formed through the rounded base 30 of the threaded cup 16. The engagement openings 50 may be configured to receive additional mating features of the wedge/spacer. The engagement openings 50 may be threaded round openings, for example.

The rounded base 30 may include one or more additional engagement openings 52 formed therethrough. The engagement openings 52 may be configured to receive mating features of an inserter device that can be utilized to implant the threaded cup 16 within the humerus 14. The engagement openings 52 may be oblong or round, for example.

The threaded cup 16 may additionally be equipped with a plurality of pockets 54. The pockets 54 may be formed in the radially outer surface 38 of the cylindrical shaped body 26 at a location just inward of the flange 28. The pockets 54 may facilitate bony ingrowth post-insertion. Alternatively or additionally, a porous coating could be applied to select portions of the cylindrical shaped body 26 for facilitating bony ingrowth. In yet another embodiment, a surface finish of the thread 36 may be grit blasted to promote bony ingrowth. The pockets 54 may be oblong shaped, in an embodiment.

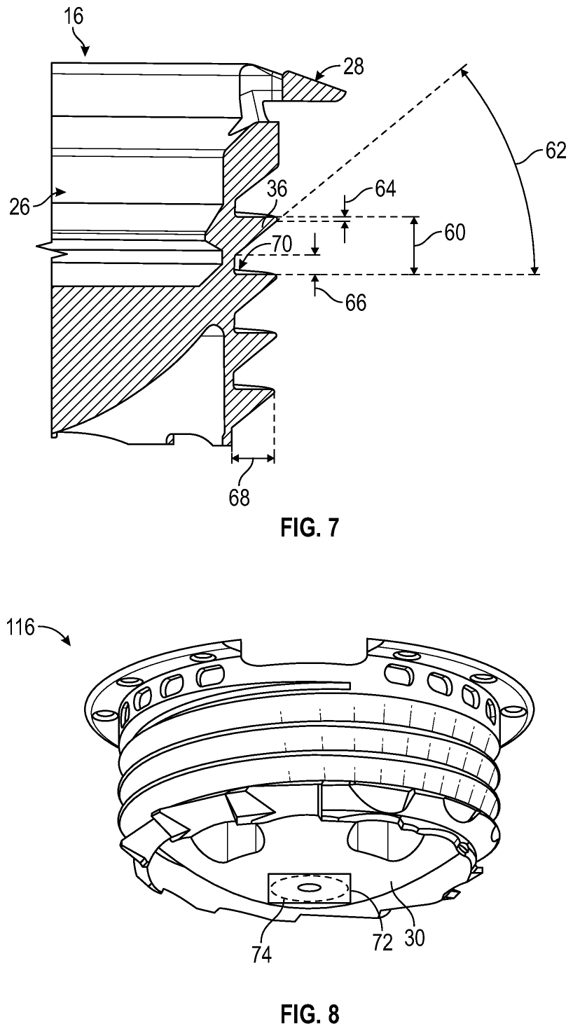

FIG. 7 illustrates additional details associated with the thread 36 of the threaded cup 16. The thread 36 may include design characteristics such as a thread pitch 60, a thread angle 62, a thread tip width 64, a thread root width 66, a thread depth 68, and a thread root radius 70. Table 2, provided below, illustrates exemplary design characteristics of the thread 36. The disclosed characteristics are intended to be exemplary only, and thus other thread specific formulations are contemplated as within the scope of this disclosure. In this disclosure, the term “about” means that the expressed quantities or ranges need not be exact but may be approximated and/or larger or smaller, reflecting acceptable tolerances, conversion factors, measurement error, etc.

TABLE 2
Exemplary Design Characteristics of Thread 36

| Thread Property | Dimension |
|---|---|
| Thread Pitch | About 2.70 mm |
| Thread Angle | About 40° |
| Thread Tip Width | About 0.19 mm |
| Thread Root Width | About 0.83 mm |
| Thread Depth | About 2 mm |
| Thread Root Radius | About 0.20 mm |

The thread pitch 60 may be a variable pitch. In an embodiment, the variable pitch increases in a direction that extends from the proximal side of the threaded cup 16 toward the distal side of the threaded cup 16. In another embodiment, the variable pitch increases in a direction that extends from the distal side of the threaded cup toward the proximal side of the threaded cup 16.

The thread 36 may be a single lead thread, a double lead thread, or a triple lead thread. The thread lead may be modified to control the amount of turns it takes to seat the threaded cup 16 within bone.

The threaded cup 16 may be implanted into the humerus 14 during a shoulder arthroplasty procedure. The shoulder arthroplasty procedure may include at least the following steps: (1) prepare the humerus 14 for receiving the threaded cup 16 (e.g., resect humeral head, prepare cavity within resected humerus, etc.); (2) screw the threaded cup 16 into the prepared humerus; and (3) connect an articular implant 18A, 18B to the implanted threaded cup 16. A greater or fewer number of steps may be performed as part of the shoulder arthroplasty procedure within the scope of this disclosure. Once implanted, the thread 36 of the threaded cup 16 may engage the humerus 14 near the cortical bone 42, and the flange 28 may load against the cortical rim 40. The thread 36 may therefore engage the cortical bone 42, cancellous bone 43, or both.

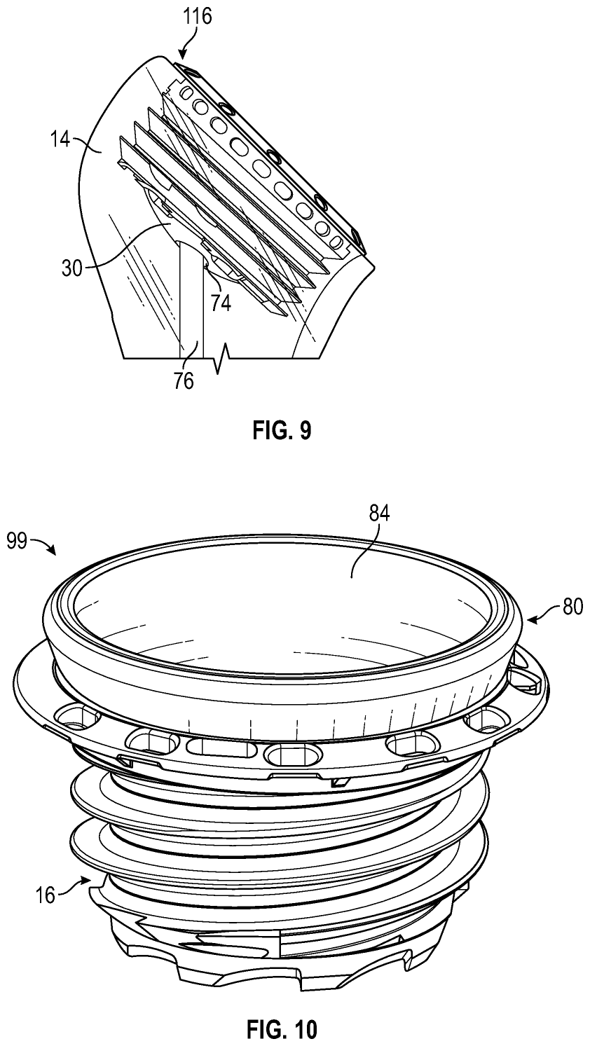

FIGS. 8 and 9 illustrate another exemplary threaded cup 116 for a humeral implant assembly of an arthroplasty implant system. The threaded cup 116 is similar to the threaded cup 16 described above. However, in this embodiment, the threaded cup 116 may include a trap door 72 that may be movably or removably connected to the rounded base 30 of the threaded cup 116. The trap door 72 may be moved relative to or removed from the rounded base 30 to expose an opening 74 through the rounded base 30. Another implant, such as a stem 76 (see, e.g., FIG. 9), may be inserted through the opening 74 and connected to the threaded cup 116 when the trap door 72 is moved/removed for improving the torsional stability of the threaded cup 116.

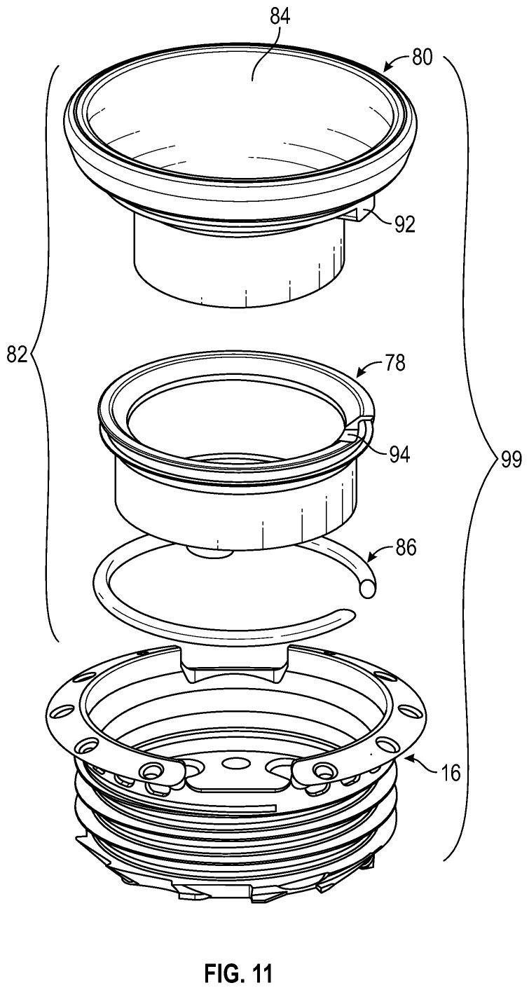

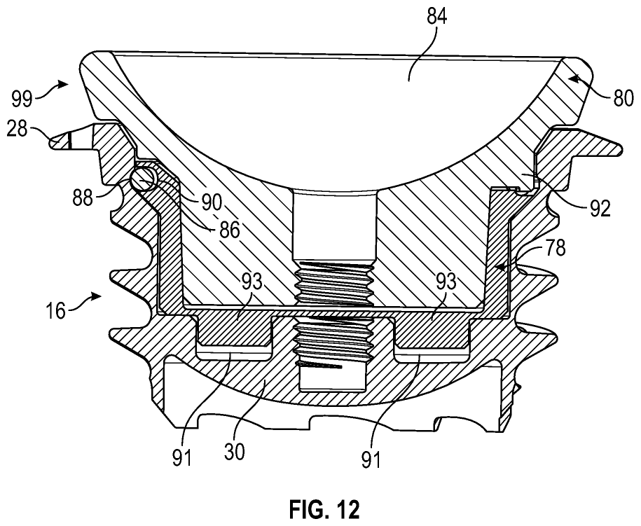

Referring now to FIGS. 10, 11, and 12, the threaded cup 16 (or the threaded cup 116) may be utilized as part of a humeral implant assembly 99 of an arthroplasty implant system. In addition to the threaded cup 16, the humeral implant assembly 99 may include a spacer 78 and a liner 80. Together, the spacer 78 and the liner 80 may establish an articular implant 82 of the humeral implant assembly 99.

In this embodiment, the articular implant 82 is a reverse articular implant. Therefore, the liner 80 may include a concave articular surface 84. However, anatomic articular implants are also contemplated within the scope of this disclosure (see, e.g., FIG. 1).

The threaded cup 16 may include one or more engagement openings 91 formed in the rounded base 30. The engagement openings 91 may accommodate de-rotation pegs 93 of the spacer 78 (or of the liner 80 if the spacer 78 is not used) for rotationally stabilizing the spacer 78 relative to the threaded cup 16.

A C-clip 86 may be used to couple the spacer 78 to the threaded cup 16, and the liner 80 may be coupled to the spacer 78 by a taper connection or any other connection. The C-clip 86 may be accommodated within a circumferential groove 88 formed in the receiving portion 32 of the threaded cup 16, and the C-clip 86 may be further accommodated within a circumferential groove 90 formed in the spacer 78.

In an embodiment, the liner 80 includes a lock block 92. The lock block 92 may be accommodated within a notch 94 formed in the spacer 78. The lock block 92 is configured to prevent the C-clip 86 from deforming inwards and allowing the spacer 78 to disengage from the threaded cup 16.

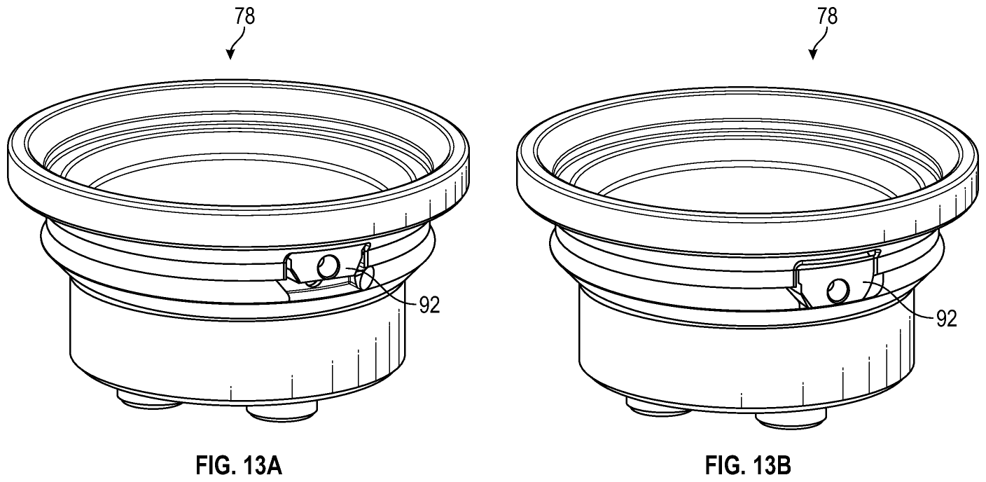

In another embodiment, the lock block 92 is provided by the spacer 78 (see, e.g., FIGS. 13A and 13B). In this implementation, the lock block 92 may be translated between an open position (FIG. 13A) in which the C-clip 86 is free to deform, and a locked position (FIG. 13B) in which the C-clip 86 is prevented from deforming. The lock block 92 may move from the open position to the locked position in response to a force applied by the liner 80 as the liner is moved into coupling engagement with the spacer 78.

In other implementations, the spacer 78 may be secured to the threaded cup 16 via a taper connection. In still other implementations, the spacer 78 may be eliminated from the humeral implant assembly 99, and the liner 80 may be directly secured to the threaded cup 16, such as via either a taper connection or a C-clip, for example. Thus, this disclosure is not intended to be limited to the exact implementations shown in FIGS. 10-13B.

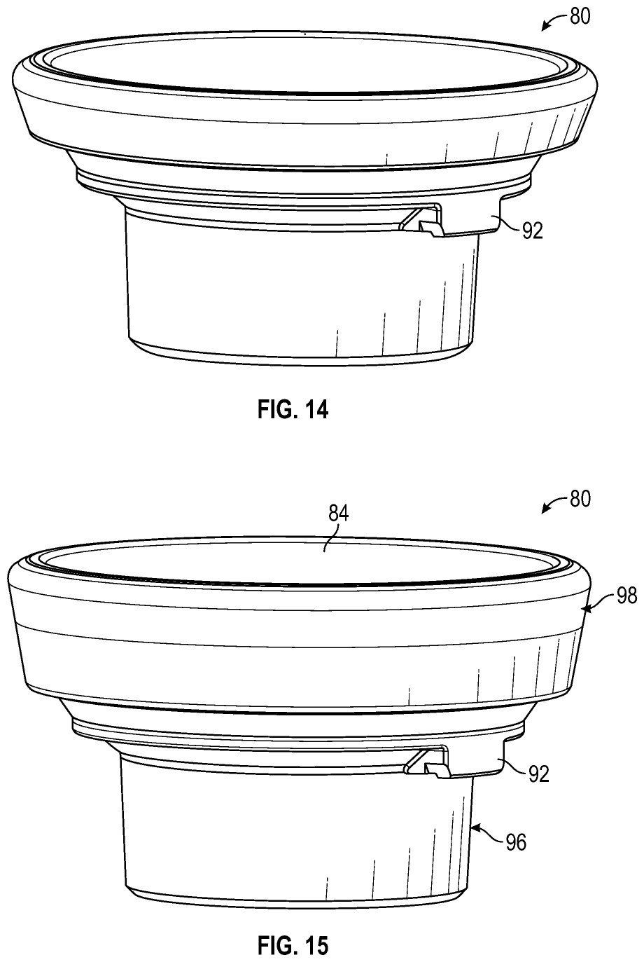

In an embodiment, the liner 80 is a metallic component (see FIG. 14). In another embodiment, the liner 80 may include both a metallic portion 96 and a polymeric portion 98 (see FIG. 15). The polymeric portion 98 may establish the concave articular surface 84 of the articular implant 82 and may be insert molded into a shell provided by the metallic portion 96.

The exemplary arthroplasty implant systems of this disclosure employ stemless implants capable of establishing a convertible platform for interfacing with articular implants. The stemless implants may be configured as threaded cups that incorporate a flange (e.g., a circumferential ring/trunnion) that is adapted to rest atop the cortical rim of a resected bone in order to provide additional fixation support and load bone more favorably when implanted. The stemless implants may further provide inlaid designs that allow for inlay reverse prosthesis configurations.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A humeral implant assembly for an arthroplasty implant system, comprising:
an articular implant; and
a stemless implant adapted to establish a convertible platform for receiving the articular implant;
wherein the stemless implant includes a thread configured to engage a bone and a flange sized to engage a cortical rim of the bone.

2. The assembly as recited in claim 1, wherein the articular implant includes a spacer that is coupled to the stemless implant and a liner that is coupled to the spacer, and further wherein the liner includes a concave articular surface.

3. The assembly as recited in claim 2, wherein the spacer is coupled to the stemless implant by a C-clip.

4. The assembly as recited in claim 1, wherein the stemless implant is comprised of a polyether ether ketone (PEEK) material.

5. The assembly as recited in claim 1, wherein the stemless implant includes a receiving cavity adapted to receive the articular implant, and further wherein the receiving cavity extends inwardly from the flange to a floor of a rounded base of the stemless implant.

6. The assembly as recited in claim 5, wherein the floor establishes an inner surface of the rounded base.

7. The assembly as recited in claim 1, wherein the thread is circumferentially disposed about a radially outer surface of a cylindrical shaped body of the stemless implant.

8. The assembly as recited in claim 7, wherein an outer diameter of the flange is greater than an outer diameter of the cylindrical shaped body at a tip of the thread.

9. The assembly as recited in claim 7, wherein the cylindrical shaped body includes a plurality of pockets adapted to facilitate bony ingrowth.

10. The assembly as recited in claim 1, wherein the flange includes a plurality of suture eyelets that are each configured to receive a thread-like material.

11. An arthroplasty implant system, comprising:
a threaded cup including a cylindrical shaped body, a thread provided on the cylindrical shaped body, and a flange,
wherein the thread is configured to engage a bone and the flange is sized to engage a cortical rim of the bone.

12. The system as recited in claim 11, wherein the flange is integrally formed with the cylindrical shaped body to establish a unitary single piece design of the threaded cup.

13. The system as recited in claim 11, wherein the threaded cup embodies an inlay design that establishes a convertible platform for receiving an articular implant.

14. The system as recited in claim 13, wherein the articular implant includes a spacer coupled to the threaded cup by a C-clip, and a liner coupled to the spacer.

15. The system as recited in claim 11, wherein the flange is provided on a first side of the cylindrical shaped body and a rounded base is provided on a second side of the cylindrical shaped body.

16. The system as recited in claim 15, wherein a receiving cavity extends inwardly from the flange to a floor of the rounded base, and further wherein the floor establishes an inner surface of the rounded base.

17. The system as recited in claim 15, comprising at least one engagement opening formed through the rounded base.

18. The system as recited in claim 15, wherein the rounded based includes a trap door.

19. The system as recited in claim 11, wherein an outer diameter of the flange is greater than an outer diameter of the cylindrical shaped body at a tip of the thread.

20. The system as recited in claim 11, wherein the cylindrical shaped body includes a plurality of pockets adapted to facilitate bony ingrowth, and further wherein the flange includes a plurality of suture eyelets that are each configured to receive a thread-like material.
